(19) United States
(12) Patent Application Publication
(10) Pub. No.: US 2015/0148555 A1
(43) Pub. Date: May 28, 2015

(54) Method for Solubilising and Separating One or a Plurality of Carboxylic Acids and Use of a Demobilised Solubilising Compound

(71) Applicants: Steffen Hruschka, Oelde (DE); Wladislawa Boszulak, Oelde (DE)

(72) Inventors: Steffen Hruschka, Oelde (DE); Wladislawa Boszulak, Oelde (DE)

(73) Assignee: GEA Mechancal Equipment GmbH, Oelde (DE)

(21) Appl. No.: 14/404,507

(22) PCT Filed: May 24, 2013
(86) PCT No.: PCT/EP2013/060777

§ 371 Date: Nov. 28, 2014

(30) Foreign Application Priority Data
May 30, 2012 (DE) 10 2012 104 668.9

(51) Int. Cl. C07C 51/50

(52) U.S. Cl. CPC C07C 51/50
USPC 554/185

(57) ABSTRACT

A method for solubilising and separating one or a plurality of carboxylic acids or carboxylic acid derivatives from an aqueous or organic solution, emulsion or suspension, involves (i) preparing the solution or emulsion or suspension with the carboxylic acid or the carboxylic acid derivatives; (ii) adding a quantity of a solubilising compound, in particular arginine or an arginine derivative, to the solution, suspension or emulsion in the presence of a minimum amount of water, a demobilised solubilising compound being used as a solubilising compound; (iii) separating the solubilised carboxylic acids or carboxylic acid derivatives as a carboxylic acid phase or carboxylic acid derivative phase from the solution or emulsion or suspension; and (iv) preferably regaining at least one portion of the solubilising compound or one portion of a derivative of the solubilising compound, in particular of the arginine or the arginine derivative, from the solubilised and separated carboxylic acid phase or carboxylic acid derivative phase.

### BACKGROUND AND SUMMARY OF THE INVENTION

Exemplary embodiments of the invention relate to a method for solubilizing and separating one or a plurality of carboxylic acids and to the use of a demobilised solubilizing compound.

PCT patent document WO 2011/160857 discloses such a method in which an arginine-water solution is particularly preferably used for solubilizing or catalysis.

In terms of the details, particularly with respect to the definition of arginine and derivatives thereof, reference is made extensively to the disclosure of WO 2011/160857, particularly pp. 24-26 of WO2011/160857.

A problem with the method, not yet solved, is the relatively high cost of providing the solubilizing compound (catalyst), particularly the arginine for preparing the arginine-water solution.

Exemplary embodiments of the invention address this problem.

In accordance with the invention, the arginine (Arg) is wholly or partly recovered here.

Analogously to the method known from WO2011/160857, a solution or emulsion or suspension of carboxylic acids in oil is provided in a first step, which are known in detail from WO 2011/160857. Subsequently, according to the invention, an amount, preferably—but not necessarily—of an at least equimolar amount of at least one solubilizing compound, particularly arginine or an arginine derivative, is added to the solution, suspension or emulsion.

Arginine is understood to mean here both L-arginine and D arginine or also a racemic mixture of both compounds, and also derivatives thereof. For further definition of arginine or derivatives thereof, reference is made to pp. 24-26 of WO2011/160857. In contrast to WO2011/160857, however, the arginine is added in the form of an immobilized compound.

In this case, the pH is adjusted to above 9, preferably to pH=11, and a micelle of water microdroplets is formed around the immobilized arginine or derivative thereof. These water micelles may settle out from the oil phase and may be separated by centrifugation as a water phase.

Subsequently, the carboxylic acid or the carboxylic acid derivatives are solubilised and the solubilized carboxylic acids or derivatives are separated as a corresponding phase from the solution, emulsion or suspension. A solubilizing compound in the scope of this application is referred to as a catalytic compound or as solubilizing a catalytic process.

Finally, at least a portion of the solubilizing compound or a portion of a derivative of the solubilizing compound, particularly the arginine or the arginine derivative, is preferably recovered from the solubilized and phase-separated carboxylic acids or carboxylic acid derivatives.

This multiple use and recovery of arginine enables efficient use of the arginine and reduces the total cost of the process.

It is of advantage when the pH is altered to a value below 7.5 in the recovery process. By lowering the pH, the bond between the carboxylic acid and the arginine is so weakened that an additional improvement in the separation and recovery of the arginine, preferably by filtration or centrifugation, is possible. In this context, a derivative may be, for example, a protonated species of arginine.

It is advantageous if the solubilizing compound used is an immobilized solubilizing compound. The recovery is thereby considerably simplified since, due to the location of the solubilizing compound, a heavier component is created that can be better removed by centrifugal separation from the carboxylic acid phase or solution.

The immobilized solubilizing compound in this case has a reactive component that enables the solubilization of a carboxylic acid, preferably a fatty acid.

This reactive component is preferably arginine or an arginine derivative. Reactive components of this kind that are useful for a solubilizing compound are adequately described in the section “Solvation and adhesion behaviour of fatty acids in aqueous media” in WO 2011/160857.

In contrast to this, an immobilized solubilizing compound preferably has, in addition, a support material to which the solubilizing compound has been applied.

This support material is preferably of a granular or spherical configuration in order to afford the largest possible surface area for the deposition of the reactive component.

### DETAILED DESCRIPTION

Based on WO2011/160857, a water/arginine solution is preferably added to an oil having free fatty acids and phosphatides. The arginine or arginine derivative has a pH of at least 9, preferably 11. After centrifuging the mixture, an arginine-fatty acid-phosphatide-water phase remains as a heavy phase from which, in addition to the free fatty acid, also the arginine is to be processed.

In this case, water could be evaporated to recover the arginine, in accordance with WO 2011/160857, which is cost intensive. Alternatively, the arginine could be filtered off from an immobilized catalyst using a sieve. For the sieving or filtration, the arginine could be agglomerated to form larger units.

The arginine may preferably be recovered, in accordance with first preferred reaction conditions, by lowering the pH by addition of an acid, preferably phosphoric acid. In this case, the pH is preferably lowered to less than 2, particularly to pH=1 or less.

After lowering the pH, a continuous water phase results having suspended, finely-dispersed arginine on the one hand and a jelly of free fatty acid and phosphatides on the other.

Subsequently, a separation may be carried out, by filtration for example, wherein an aqueous arginine phase remains. Subsequent to this, the pH is then in turn adjusted by neutralization, preferably with aqueous sodium hydroxide, particularly with NaOH, to the alkaline range of over pH=9, preferably to pH 11. The resulting salt should be separated off.

In accordance with second preferred reaction conditions for the recovery of the arginine, alcohol is added to the arginine-fatty acid-phosphatide-water phase and the pH is adjusted to less than 7.5, particularly to pH=5-6.

The free fatty acid-phosphatide phase can then be centrifuged off as a light phase. An aqueous alcoholic arginine solution remains having a pH of preferably 5-6, from which the alcohol should be removed. The pH is then in turn adjusted to a pH of over 9, preferably to pH=11.

By means of the abovementioned variants for the separation of the arginine, said arginine is available as solubilizing compound for further solubilizations of carboxylic acids.

These two and other possible variants always lead, however, to an aqueous finely dispersed arginine with relatively large amounts of water. The abovementioned reaction conditions of the first and second embodiment variants represent preferred reaction conditions of the following method according to the invention. However, the requirement for large amounts of liquid, acids, aqueous sodium hydroxide solution and the resulting dilute arginine solution is disadvantageous.

It is therefore an advantage of the present invention that the arginine is to be recovered relatively dry from the water phase, particularly preferably by centrifugal separation.

With particular preference, this is achieved by the division into

a) a free fatty acid-phosphatide phase

b) a water phase

c) an arginine-support phase (arginine granulate).

For this purpose, an arginine granulate is provided according to an advantageous further development of the method according to the invention, which may then be separated as a relatively dry arginine-support phase c). It is therefore particularly advantageous for recycling to put arginine or an arginine derivative in any form onto a solid support, whereby the arginine is immobilized.

For this purpose, arginine is preferably applied to PVPP (polyvinylpyrrolidone), particularly Divergan, according to the product specifications of BASF at the time of the application, prior to adding the carboxylic acid, particularly the free fatty acid. This is preferably carried out by spraying the aqueous arginine onto the PVPP support material.

In this case, the carboxyl group of the arginine adheres to the microscopically scored PVPP, preferably Divergan F, and the guanidine group remains as active group, for reaction with the fatty acid, on the surface of the support material. The pH of the amino acid is preferably in this case between 9-10.

Subsequently, the arginine applied to the support material is added to the oil or to the carboxylic acid solution, suspension or emulsion. This addition is carried out by addition of water. Here, the free fatty acid is solubilized, as already described above, and is removed from the solution as free fatty acid-phosphatide phase a), preferably by centrifugation.

The presence of a minimum amount of water is essential here for the solubilization, as otherwise no separation of the fatty acids from an oil by means of the solubilizing compound would be possible. The water may already be present in part in the oil, for example, as a colloidal solution. Should the amount not be sufficient, additional water can still be added.

After or at the same time as the separation of the free fatty acid-phosphatide phase a), the aqueous granules or the support material provided with arginine are separated. Here, the pH should preferably be reduced to less than 7.5, preferably 5-6, so that the free fatty acids (FFA) lose their binding to the arginine and the arginine-PVPP granulate may be separated. This may optionally be carried out with the help of alcohol, as has already been explained in the above-described second embodiment example.

After washing and, if necessary, drying (even partially), the arginine immobilized on the support material can be reused.

Particularly preferred here is the presence of water. The sole addition of the granules in an oil is not sufficient; it must be dissolved in water or at least be suspended. A separation of the arginine granulate with the free fatty acids and the phosphatide groups from an oil phase is thereby possible.

On adjusting the water phase according to the abovementioned preferred first reaction conditions, the complexes of PVPP-arginine, the free fatty acids and the phospholipids combine with water to form a micelle in the oil phase or water microdroplets, which settle out from the oil phase, and a water phase forms that can be centrifuged off

On adjusting the water phase according to the abovementioned preferred second reaction conditions, for example for a 15% ethanol solution at pH=5.0, this also results in a fine separation into a phospholipid/fatty acid phase and an immobilized arginine phase, wherein the arginine-PVPP phase settles out as a heavier phase from the phospholipid/fatty acid mixture.

Thus, in addition to the water phase, after the separation of the oil phase, there arises a three phase system of a clear aqueous ethanolic solution, a mixture of phospholipids and free fatty acids and the arginine-PVPP phase, which may be separated as a cake with a low moisture content.

As an alternative to immobilizing the arginine on a support material, the immobilization of the arginine, in a second embodiment variant of the method according to the invention, can also be carried out by conversion into an arginine derivative—preferably a peptide—and subsequent application to a support material.

In this case, a peptide from arginine is firstly prepared by adding a preferably sulfur-containing amino acid such as cysteine or methionine. This diaminopeptide, in which the guanidine group of the resulting peptide binds to the free fatty acid, can be deposited on a support material—preferably on steel beads. The iron in the steel binds to the functional groups of the amino acid, preferably the sulfur-containing amino acid. A steel-amino acid-arginine complex is thus formed to which water is also added, where this is not already present suspended in the oil phase. Here, once again, a micelle forms from the complex of the free fatty acid, phospholipid and water, which settles out in the oil phase and forms an aqueous phase.

As already described, the formation of the complex can be specifically controlled if the arginine-amino acid complex is first formed and this is subsequently applied to the surface of the steel beads. Here, the amino acid forms the bond between the respective steel bead and the arginine molecule without influencing the functionality of the arginine.

The abovementioned first or second reaction conditions may also preferably be applied to this embodiment variant, in which, in the second reaction condition, for example, in a 15% ethanol solution at pH=5, a sedimentation of the complex of the steel beads from the mixture of free fatty acids and phospholipids occurs. After the 3-phase separation, a clean aqueous ethanolic solution, a phospholipid-fatty acid solution and a cake of the complex with low residual moisture content are obtained.

The addition and recovery of this combination of the arginine derivative and the support material in the solubilization method is carried out analogously to the abovementioned embodiment variant of the PVPP granulate.

Investigations relating to the second embodiment variant of the immobilization have shown that the duplex and ferritic steels have a much better protein adhesion than austenite. In addition, the adhesion is dependent on the different composition of the proteins. Thus, some amino acids adhere better than others. Important in this context is the HS group or the sulfur group of the proteins.

This preferably binds to the iron of the steel. It has also been found that nitrogen-containing steels provide better adhesion for the abovementioned arginine derivatives than steels having a negligibly small amount of nitrogen. Therefore, the use of nitrogen-containing steels as support material is preferred.

The surface structure of the steals also plays a role. After the hot rolling of the steels, they are pickled such that holes or pores less than 1 micrometer (microporosity) are formed on the surface of the steels into which peptides or amino acids can accumulate. Therefore, preference is given to using steels as support materials having microporous structures, i.e. structures having holes with a mean diameter of less than 1 micrometer. In particular, the use of hot-rolled steels.

It is also possible, moreover, to initially immobilize suitable amino acids on a metal—particularly steel surface and then to provide a corresponding arginine derivative by adding the arginine.

The combination of suitable amino acids or peptides, cysteine, or thiamine for example, having good adhesion to the abovementioned, preferably nonaustenitic, steels, with functional amino acids, particularly arginine, or peptides, particularly enzymes, permits simple recovery and multiple use of the solubilizing compound, i.e. the arginine or the arginine derivative respectively.

Accordingly, these combination products can then be allowed to adhere to the steels or steel powders having suitable microporous surfaces. Steel-weighted functional products such as amino acids are thus obtained, which are stuck to steel surfaces and thus are specifically heavier than oil or water and may be readily separated by centrifugation from the abovementioned phase mixture.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof

1-13. (canceled)

14. A method for solubilizing and separating one or more carboxylic acids or carboxylic acid derivatives from an aqueous or organic solution, emulsion or suspension, comprising the following steps:
i) providing the solution, emulsion, or suspension with the carboxylic acid or the carboxylic acid derivatives;
ii) adding an amount of a solubilizing compound to the solution, suspension, or emulsion in the presence of a minimum amount of water, wherein the solubilizing compound used is an immobilized solubilizing compound, and wherein the solubilizing compound is arginine or an arginine derivative;
iii) separating the solubilized carboxylic acids or carboxylic acid derivatives from the solution, emulsion, or suspension as a carboxylic acid or carboxylic acid derivative phase; and
iv) recovering at least a portion of the solubilizing compound or a portion of a derivative of the solubilizing compound from the solubilized and separated carboxylic acid phase or carboxylic acid derivative phase.

15. The method of claim 14, wherein the recovery in step iv) involves changing the pH of the solubilized carboxylic acids or carboxylic acid derivative phase to a pH of less than 7.5.

16. The method of claim 15, wherein the immobilized solubilizing compound has a reactive component that enables the solubilization of a fatty acid, and has a support material to which the solubilizing compound is applied.

17. The method of claim 16, wherein the support material is a granulate of PVPP, zeolite, kieselguh, or another diatomaceous earth or bentonite.

18. The method of claim 16, wherein the reactive component of the immobilized solubilizing compound is a peptide prepared from arginine or an arginine derivative and a sulphur-containing amino acid.

19. The method of claim 16, wherein the support material is a metal.

20. The method of claim 16, wherein the support material is a steel.

21. The method of claim 16, wherein the support material is a nonaustenitic steel.

22. The method of claim 16, wherein the reactive component is an arginine or an arginine derivative.

23. The method of claim 14, wherein the solubilized carboxylic acid or carboxylic acid derivative phase is separated from the solution, emulsion, or suspension in the form of an arginine-fatty acid-phosphatide-water phase.

24. The method of claim 23, wherein the bond between the carboxylic acid or the carboxylic acid derivatives and the solubilizing compound is weakened or broken by reducing the pH to less than 7.5.

25. The method of claim 23, wherein the bond between the carboxylic acid or the carboxylic acid derivatives and the solubilizing compound is weakened or broken by reducing the pH to a pH=5-6.

26. The method of claim 24, wherein the pH is reduced up to a maximum pH=2.

27. The method of claim 24, wherein a polar solvent is added during the lowering of the pH.

28. The method of claim 14, wherein the recovery according to step iv is carried out by centrifugal separation of the immobilized, solubilized compound from the solubilized and separated carboxylic acid phase or carboxylic acid derivative phase.
